(19) United States
(12) Patent Application Publication
(10) Pub. No.: US 2023/0132481 A1
(43) Pub. Date: May 4, 2023

(54) HYPODERMIC NEEDLE FOR INTRA-ARTERIAL/INTRAVENOUS LINE PLACEMENT

(71) Applicant: Satnam Ludder, Modesto, CA (US)

(72) Inventor: Satnam Ludder, Modesto, CA (US)

(21) Appl. No.: 17/515,470

(22) Filed: Oct. 31, 2021

(51) Int. Cl. A61M 25/06; A61M 25/00; A61M 25/09

(52) U.S. Cl. CPC A61M 25/06; A61M 25/0097; A61M 25/09

(57) ABSTRACT

An improved hypodermic needle featuring a tip with an aperture that is not axially aligned with the shaft of the needle designed to increase the success rate of needle insertion for placing a guidewire by reducing the incidence of complications such as vessel wall injury or dissection and distal embolization and providing the physician with greater feedback confidence by giving more accurate bleed-back from the true lumen of the vessel.

### FIELD OF THE PRESENT DISCLOSURE

This disclosure relates generally to medical equipment and more specifically to an improved hypodermic needle fashioned to increase the success rate and safety of needle insertion for the purpose of placing an intra-arterial/intravenous line (IA/IV line) by eliminating common challenges and providing physicians with greater feedback confidence and reducing complications.

### BACKGROUND OF THE RELATED ART

There are a variety of medical reasons why a physician may decide to place an intra-arterial/intravenous line in a patient. Such reasons range from the need to perform relatively simple procedures such as the placement of a cannula for administering medications or fluids intravenously to more complex and involved endovascular procedures such as coronary angioplasty and stent placement or peripheral artery disease intervention, to name a few.

In each of these procedures, vascular access is traditionally gained via the insertion of a legacy needle guided only by palpation of the pulse and sometimes also an anatomic landmark identification technology such as fluoroscopy or ultrasound. However, in all cases, regardless of whether or not an assistance technology is utilized, the tactile feel, visual feedback, and experience of the physician are of paramount importance to successfully achieving accurate needle insertion.

The legacy needles typically utilized comprise an 18 or 21 gauge shaft with a sharpened beveled tip featuring an axially aligned aperture. When placing an IA/IV line using such legacy needles, the physician must manually insert the needle in the patient at a shallow angle toward the intended vessel until the physician believes the needle tip has penetrated the proximate wall of the intended vessel but has not yet penetrated the distal wall of the intended vessel such that the needle's aperture is located wholly within the lumen of the intended vessel.

Ideally, the legacy needle is successfully inserted into the intended vessel to the appropriate depth on the first try so the physician may then advance a guidewire through the shaft of the needle and out of the needle's aperture so that the distal end of the guidewire is located in the lumen of the vessel. Once the guidewire is sufficiently advanced, the physician may then withdraw the needle leaving the guidewire in place so that it may be used as a guide to assist and direct the insertion of subsequent medical devices necessary for the particular procedure intended to be performed.

In practice however, a variety of complications can arise quite easily with legacy needles. For example, one key feed-back indicator that informs the physician when the needle has been inserted to the proper depth is the visible presence of blood flowing back up the needle which occurs when the needle's aperture pushes through the proximate wall of the intended vessel and reaches the pressurized blood flow of the vessel lumen. However, in legacy needles, because of the elongated oval shape of the axially aligned aperture due to the angle of the beveled tip, a portion of the aperture often reaches the blood flow before the needle aperture has fully penetrated the proximate vessel wall and is properly positioned within the vessel lumen. This can allow back-bleed to initiate before the needle is properly positioned inside the true lumen of the vessel.

If a physician is misled by such pre-mature back-bleed, the physician may incorrectly believe that the needle is properly positioned and begin advancing the guidewire through the needle before the needle is fully inserted causing the line to advance into the vessel wall rather than into the lumen of the vessel lumen. Depending on the force used to advance the guidewire and by the time an error is detected, the physician may damage the vessel wall intima causing at times serious dissection which can sometimes compromise the vessel lumen and can extend up or down the vessel. This can lead to loss of access site and further attempts at that site may have to be aborted. The occurrence of this particular complication can be minimized by using a needle that is less susceptible to premature bleed-back.

Another common complication associated with the legacy needle is called coring. Coring occurs when fat and/or other tissue occludes the tip of the needle during needle insertion through skin and subcutaneous tissue. When the needle tip becomes occluded or partially occluded during insertion it can block the bleed-back indication that the physician is expecting and sometimes cause the physician to continue inserting the needle past the vessel lumen and into and/or through the vessel's distal wall thus causing unnecessary vessel damage of another kind. Depending on the extent of the additional unnecessary damage to the distal vessel wall, this complication may also require the physician to abort the procedure and begin anew in a different location.

Alternatively, rather than aborting an attempt because an over-insertion error occurred, the physician may decide to back the needle out and try to salvage the effort; however, if the needle is still occluded preventing back-bleed indication, the physician may still be denied visual confirmation of when the needle is properly positioned and may have to rely on tactile feel and experience alone to properly position the needle which can sometimes result in the inadvertent advancing of the guidewire into either the proximal or distal vessel wall intimae causing vessel wall injury.

What's more, when coring occurs, even if the physician correctly positions the needle and avoids damaging the vessel walls, the physician must still contend with the additional complication of where the piece of cored tissue will ultimately be deposited after it is dislodged from the needle into the vessel lumen by the advancing guidewire. This may lead to distal embolization of the stuck material which can sometimes lead to serious complications depending upon the site of embolization.

Depending on the condition of the patient and/or the necessary location of the procedure, an aborted attempt may be a very serious error because sometimes the procedure may be critical to saving the life of the patient and the physician may not have many viable alternative locations in which to properly execute the procedure. For this reason, any design improvement that can increase the success rate of needle insertion and/or reduce the incidence of aborted attempts and vessel damage should be considered a very important potentially life-saving innovation. It will also decrease injury to surrounding tissue as it will more likely spread the tissue going in rather than cut through it as a sharp hypotube as the current needles do.

The present disclosure distinguishes over the related art providing heretofore unknown advantages as described in the following summary.

### BRIEF SUMMARY OF THE INVENTION

The present disclosure describes an improved innovative hypodermic needle apparatus designed to increase the accuracy and success rate of needle insertions for the purpose of placing IA/IV lines achieved by eliminating common challenges and providing physicians with greater feedback confidence through innovative and novel design features.

Similar to legacy hypodermic needles, the presently disclosed apparatus comprises a needle shaft with a proximal and distal terminus, a hub affixed to the proximal terminus of the needle shaft designed to facilitate a physician's grasp and manipulation, and a sharp tip affixed to the distal terminus of the needle shaft, designed to facilitate penetrating flesh, which features an aperture through which a guidewire may be advanced.

The novel innovation of the presently disclosed apparatus lies in the design of the sharp tip, specifically, in the features of the tip intended to better facilitate the line placement procedure. Similar to legacy needles, the novel apparatus can accept a guidewire advanced through the entire apparatus, entering from the proximal terminus, passing through the needle shaft, and exiting through an aperture in the sharpened tip: however, unlike legacy needles, the aperture is not aligned with the axis of the shaft and the aperture is not located at the distal most point of the sharpened tip.

These innovative design features have multiple practical benefits. One such benefit is that such features reduce the incidence of pre-mature bleed-back. This is because by placing the aperture in a non-axial orientation, the size and shape of the aperture is not directly influenced by the shape of the beveled tip or the shape of the tip otherwise. As previously explained, legacy needles typically feature apertures with an extended oval shape due to the combined effect of the aperture's axial alignment and the angle of the bevel. This extended oval shape causes the most distal edge of the oval shaped aperture to come into contact with the blood flow as soon as the needle tip first pierces the wall of vessel, well before the needle is fully inserted into the vessel lumen and well before the needle is properly positioned to begin advancing the guidewire.

In contrast, the presently disclosed novel apparatus features an aperture that is oriented in a non-axial manner and features a shape that is not dictated by the angle of the bevel such that the leading edge of the aperture is not extended excessively in the axial direction. This reduces the incidence of premature bleed-back because when this improved aperture comes in contact with the blood flow of the vessel lumen the apparatus is much closer to the optimal position to begin advancing the guidewire; therefore, it is much less likely that the physician will inadvertently advance the guidewire into the vessel wall intima and possibly cause such damage that the attempt must be aborted and/or the insertion location is rendered unusable.

Further, because the novel apparatus features an aperture that is not aligned axially with the needle shaft there is a significantly reduced chance that the aperture will become colluded with fat or other tissue during the insertion. This is because the force of the needle being inserted into the patient is not pressing tissue directly into the aperture because the aperture is not axially located. In some embodiments the aperture is located along the slope of the sharpened tip approximately 45 degrees offset from the axis of the needle shaft, whereas in other embodiments the aperture is located as much as 90 degrees offset from the axis of the needle shaft. Regardless of the offset angle, by positioning the aperture in an orientation other than directly axial to the shaft, the chances of the aperture becoming colluded by fat or other tissue during insertion is significantly reduced thereby increasing the success rate of insertion attempts by avoiding the previously described complications and potential vessel damage associated with coring.

There exists some legacy needles designed for entirely unrelated purposes, such as irrigation and/or dispensing fluids, that also feature apertures that are offset from the shaft axis, typically by ninety degrees; however, these legacy designs differ significantly from the presently disclosed apparatus in that they typically feature an internal cavity that extends though the needle shaft and dead ends past the tangentially located aperture such that it would be very difficult or impossible to advance a guidewire down the shaft and out the aperture because the leading end of the guidewire would have a tendency to pass the aperture and become trapped in the distal dead end of such legacy needles. These legacy needles cannot be used to address the challenges that the presently disclosed apparatus resolves.

In contrast, the present disclosure features a tip that is solid adjacent to the aperture on its distal edge such that an guidewire cannot be advanced through the needle shaft past the aperture. Further, in some embodiments the internal surface of the solid tip is especially shaped so as to direct the guidewire toward the aperture. However, regardless of the embodiment of the presently disclosed novel apparatus, the guidewire cannot be advanced past the aperture such that when the physician advances the guidewire through the needle shaft and the leading end of the guidewire reaches the aperture it is guided or directed toward the aperture by the shape of the internal surface of the tip, or simply left with no meaningful path forward such that the guidewire naturally exits the aperture.

Another advantage of the presently disclosed apparatus is the greater flexibility with regards to the angle of insertion and direction of line advancement that it provides. With legacy needles it is standard practice to insert the needle at a shallow angle in the direction of the desired intra-arterial/intravenous line placement. At times, the required shallow angle of insertion presents physical challenges due to the location of the desired target vessel in relation to other physical obstructions including the patient's own body. In contrast, the presently disclosed apparatus allows for insertion to occur at relatively higher angles without complicating the advancement of the guidewire because of the angle at which the guidewire exits the non-axially aligned aperture; sometimes the angle is as much as ninety degrees offset from orientation of the needle shaft. This allows the physician more flexibility and latitude when choosing acceptable insertion locations.

In fact, some embodiments facilitate antegrade insertions where the guidewire may be advanced in an acute angle from the insertion and/or allow the physician to reverse the direction of line advancement after the needle is inserted by rotating the needle after insertion and before advancing the guidewire. The presently disclosed apparatus features an directional indicator located on the hub so that the physician can always determine the current orientation of the aperture with respect to the hub, even after insertion. Such antegrade insertions may be necessary in certain cases electively or when the traditionally optimal insertion locations are limited or physically compromised.

This disclosure teaches certain benefits in construction and use which give rise to the objectives described below:

A primary objective inherent in the above described apparatus is to provide advantages not taught by the prior art;

Another objective is to provide a novel apparatus to increase the success rate of needle insertions for the purpose of placing intra-arterial/intravenous lines by minimizing or eliminating common insertion challenges;

A further objective is to provide a novel apparatus to increase the success rate of needle insertions for the purpose of placing intra-arterial/intravenous lines by providing physicians with greater feedback confidence;

A still further objective is to provide a novel apparatus capable of providing a physician greater flexibility and latitude when choosing acceptable insertion locations for placing intra-arterial/intravenous lines;

Other features and advantages of the present invention will become apparent from the following more detailed descriptions, taken in conjunction with the accompanying drawings, which illustrate, by way of example, the principles and features of the presently described apparatus.

### BRIEF DESCRIPTION OF THE SEVERAL VIEWS OF THE DRAWINGS

The accompanying drawings illustrate various exemplary implementations and are part of the specification. The illustrated implementations are proffered for purposes of example not for purposes of limitation. Illustrated elements will be designated by numbers. Once designated, an element will be identified by the identical number throughout. Illustrated in the accompanying drawing(s) is at least one of the best mode embodiments of the present disclosure. In such drawing(s):

FIG. 1 is a cross-section view of the distal portion an exemplary embodiment of the presently disclosed improved hypodermic needled shown properly inserted into a patient's vessel lumen for context;

FIG. 2 is a cross-section view of the distal portion an exemplary embodiment of the presently disclosed improved hypodermic needle featuring a “short” conically shaped needle tip with an non-axially aligned aperture for guidewire egress;

FIG. 3 is a perspective view of an exemplary embodiment of the presently disclosed improved hypodermic needle featuring an exemplar hub to facilitate the physician's grasp and manipulation:

FIG. 4 is a cross-section view of the distal portion an exemplary embodiment of the presently disclosed improved hypodermic needled featuring a beveled and angled needle tip with an non-axially aligned aperture for the guidewire egress shown properly inserted into a patient's vessel lumen for context;

FIG. 5 is a perspective view of the distal portion of an exemplary embodiment of the presently disclosed improved hypodermic needled featuring a beveled and angled needle tip with an non-axially aligned aperture for the guidewire egress;

FIG. 6 is a plan view of the distal portion of an exemplary embodiment of the presently disclosed improved hypodermic needled featuring a beveled and angled needle tip with an non-axially aligned aperture for guidewire egress illustrating that the internal surface of the tip is shaped to direct the guidewire to the aperture;

FIG. 7 is a perspective view of the distal portion of an exemplary embodiment of the presently disclosed improved hypodermic needled featuring a conically shaped needle tip with extra wide non-axially aligned aperture for the guidewire egress

FIG. 8 is a perspective view of the distal portion of an exemplary embodiment of the presently disclosed improved hypodermic needled featuring a beveled needle tip with a non-axially aligned aperture for the guidewire egress located on the beveled surface;

FIG. 9 is plan view of the distal portion of an exemplary embodiment of the presently disclosed improved hypodermic needled featuring a “long” conical shaped beveled needle tip with an non-axially aligned aperture for the guidewire egress;

### DETAILED DESCRIPTION OF THE EXEMPLARY EMBODIMENT

The above described drawing figures illustrate multiple exemplary embodiments of the presently disclosed apparatus and its many features in at least one of its preferred, best mode embodiments, which is further defined in detail in the following description. Those having ordinary skill in the art may be able to make alterations and modifications to what is described herein without departing from its spirit and scope of the disclosure. Therefore, it must be understood that what is illustrated is set forth only for the purposes of example and that it should not be taken as a limitation in the scope of the present apparatus or its many features.

Described now in detail is an improved hypodermic needle fashioned to increase the success rate of needle insertion for the purpose of placing an intra-arterial/intravenous line by eliminating common challenges and providing physicians with greater feedback confidence.

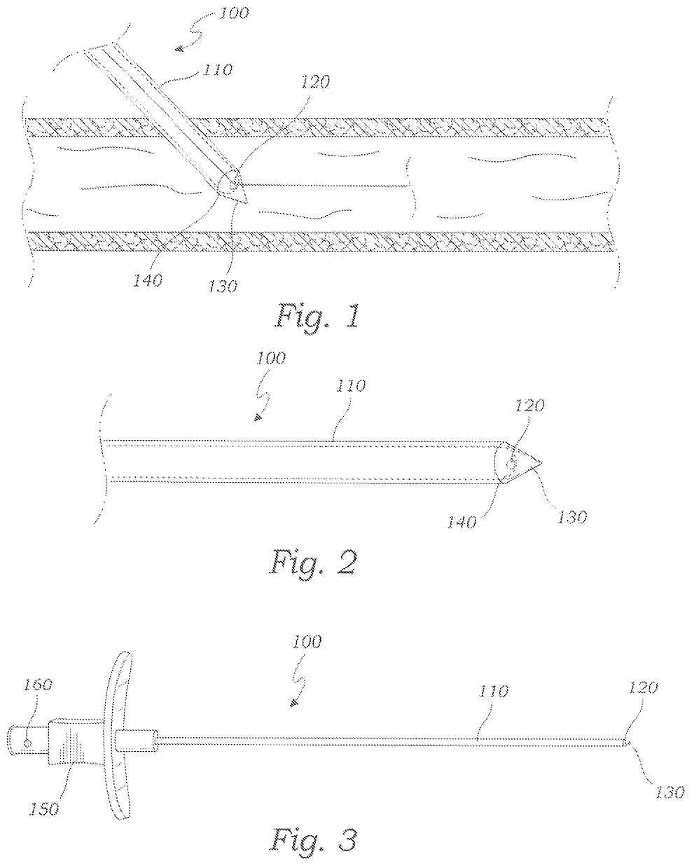

FIG. 1 illustrates an exemplary embodiment of the distal portion of the presently disclosed apparatus 100 shown properly inserted into a patient's vessel lumen for practical context. The needle shaft 110 is shown affixed to a needle tip 130 which features an aperture 120 for the egress of a guidewire, which is also illustrated for context. The particular needle tip 130 illustrated in FIG. 1 is a “short” conical shaped tip 130 designed to be sharp enough to easily penetrate the skin and subcutaneous tissue. As the solid tip is the distal most terminus of the apparatus, it will provide a more tactical feel of the arterial pulsation to assist the physician as it first touches the artery. The presently disclosed apparatus can feature a variety of needle tips 130 so long as the aperture 120 is not axially aligned to the needle shaft 110.

FIG. 2 is a perspective view the distal portion of the same embodiment featured in the contextual illustration of FIG. 1 and further illustrates features that are important to realizing the advantages of the presently disclosed innovative design such as the aperture 120 that is not axially aligned with the needle shaft 110 and the shape of the internal surface 140 of the needle tip 130 that prevents a guidewire from being advanced past the aperture 120 and/or directs the guidewire to the aperture 120. This is important because if a guidewire is allowed to be advanced past the aperture 120 then the guidewire could get trapped in the needle tip 130 or in dead end area past the aperture 120 preventing the physician from successfully advancing the guidewire through the aperture 120 and into the patient vessel lumen.

FIG. 3 is a perspective view of the entire exemplar embodiment also featured in FIGS. 1 and 2. The proximal end of the embodiment 100, not previously shown, illustrates an exemplar embodiment of the hub 150 designed to ergonomically lit the hand of the physician to facilitate the physician's grasp and manipulation. The hub 150 also features a directional indicator 160 to keep the physical apprised as to the orientation of the aperture 120 even after insertion. This is especially important during high-angle insertions or antigrade insertions where the orientation of the aperture 120 is not necessarily immediately apparent from the angle of the apparatus 100. Both the hub 150 and directional indicator 160 can take on many different forms so long as the features can serve their respective functions.

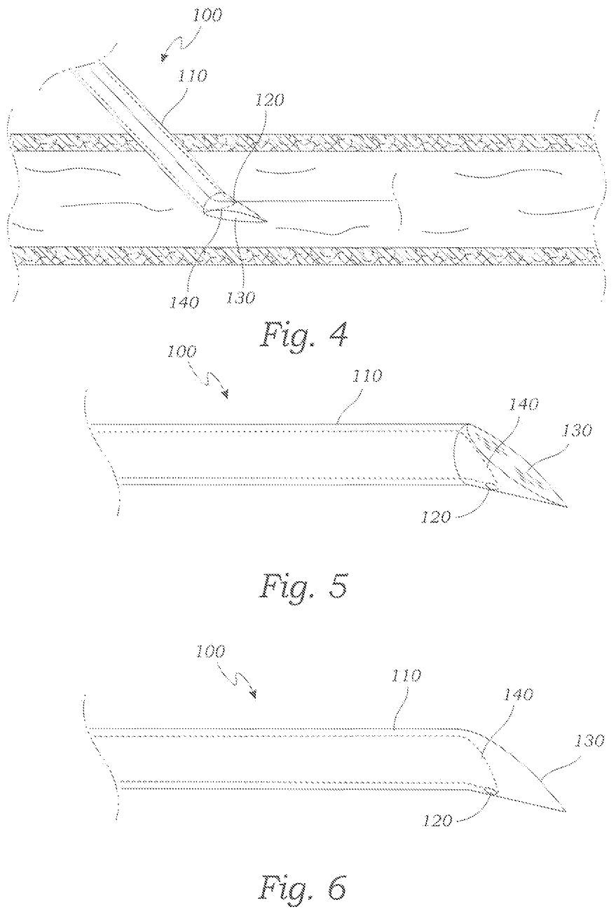

FIG. 4 is a contextual illustration similar to FIG. 1 featuring the distal portion of an exemplar embodiment inserted into a patient's vessel also depicting a guidewire advanced through a non-axial aperture. The embodiment 100 illustrated in FIG. 4 differs from the embodiment 100 illustrated in FIG. 1 in that the tip 130 in the embodiment 100 illustrated in FIG. 4 features a beveled sharpened tip 130 with an opposing non-axial aperture 120.

FIG. 5 shows a magnified partial view of the same embodiment illustrated in FIG. 4 such that it is apparent that the beveled tip 130 in this particular exemplar embodiment is affixed to the needle shaft 110 in an non-axial or angled manner. In certain circumstances, the angled mounting of the needle tip 130 may allow the physician the ability to insert the apparatus 100 at a higher angle than with legacy needles. Such embodiment variations are possible without departing from the spirit of the novel innovation or the associated advantages. FIG. 6 is a plan view that further illustrates the nonsymmetrical shape of the internal surface 140 of the tip 130 which is especially shaped to direct an advancing guidewire toward the aperture 120 thereby ensuring smooth egress.

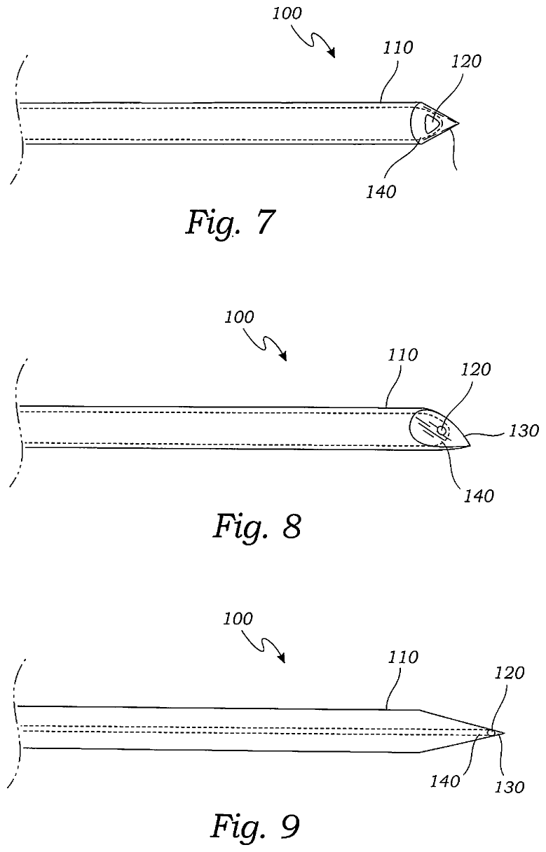

FIGS. 7 and 8 feature further variations of the presently disclosed embodiment 100, including a “short” conical tip 130 with a “wide” non-axially aligned aperture 120 (FIG. 7) and a beveled tip 130 with and aperture 120 located on the beveled surface (FIG. 8). In each embodiment, the aperture 120 in non-axially aligned with the needle shaft 110 and the internal surface 140 of the needle tip 130 is shaped such that an guidewire cannot advance through the needle shaft 110 past the aperture 120.

FIG. 9 illustrates one further exemplar embodiment in which the needle tip 130 is a “long” conical shape which is sharper and can penetrate flesh with greater easy than the “short” conical shape of the needle tips 130 featured in FIGS. 1, 2, and 7. The shape of the needle tip in the illustrative exemplar are is not indicative of limits of the presently disclosed innovation, the needle tip can take on many forms so long as the aperture 120 for guidewire egress is non-axial to the needle shaft 110 and the internal surface 140 of the needle tip prevents a guidewire from advancing through the apparatus 100 past the aperture 120. Here, the exemplar embodiment 100 illustrated in FIG. 9 features an aperture 120 that is very close to the needle tip 130 but it is still positioned non-axially to the needle shaft 110.

The enablements described in detail above are considered novel over the prior art of record and are considered critical to the operation of at least one aspect of the apparatus and its method of use, and to the achievement of the above-described objectives. The words used in this specification to describe the instant embodiments are to be understood not only in the sense of their commonly defined meanings, but to include by special definition in this specification: structure, material, or acts beyond the scope of the commonly defined meanings. Thus, if an element can be understood in the context of this specification as including more than one meaning, then its use must be understood as being generic to all possible meanings supported by the specification and by the word(s) describing the element.

The definitions of the words or drawing elements described herein are meant to include not only the combination of elements which are literally set forth, but all equivalent structures, materials or acts for performing substantially the same function in substantially the same way to obtain substantially the same result. In this sense it is therefore contemplated that an equivalent substitution of two or more elements may be made for any one of the elements described and its various embodiments or that a single element may be substituted for two or more elements in a claim.

Changes from the claimed subject matter as viewed by a person with ordinary skill in the art, now known or later devised, are expressly contemplated as being equivalents within the scope intended and its various embodiments. Therefore, substitutions, now or later known to one with ordinary skill in the art, are defined to be within the scope of the defined elements. This disclosure is thus meant to be understood to include what is specifically illustrated and described above, what is conceptually equivalent, what can be obviously substituted, and also what incorporates the essential ideas.

The scope of this description is to be interpreted only in conjunction with the appended claims and it is made clear, here, that each named inventor believes that the claimed subject matter is what is intended to be patented.

What is claimed is:

1. An improved needle apparatus for facilitating intra-arterial/intravenous line placement, said apparatus comprising:
a hub for facilitating a physician's grasp;
a needle shaft with both a proximal terminus and a distal terminus, and
a needle tip featuring an aperture for egress of a guidewire;
wherein said hub is affixed to the proximal end of said needle shaft and said tip is affixed to the distal terminus of said needle shaft such that a guidewire may ingress the proximal terminus of said needle shaft, be advanced through said needle shaft, and egress through the aperture in said tip;
wherein said aperture is non-axially aligned with said needle shaft;
wherein said needle tip is shaped such that a guidewire cannot be advanced to a more distal point than said aperture.

2. As apparatus as in claim 1 wherein said hub further comprises a directional indicator in a fixed orientation with said aperture such that the radial location of the aperture can be determined from the position of said directional indicator.
